(19) United States
(12) Patent Application Publication
(10) Pub. No.: US 2003/0065290 A1
(43) Pub. Date: Apr. 3, 2003

(54) Disposable safety syringe

(75) Inventor: Mei-Ching Shyu, Taipei (TW)

Correspondence Address: Jackson Walker L.L.P., Suite 2100, 112 E. Pecan Street, San Antonio, TX, 78205, US

(73) Assignee: Cosbin Investment Inc.

(21) Appl. No.: 09/972,238

(22) Filed: Oct. 3, 2001

(51) Int. Cl. A61M 5/00

(52) U.S. Cl. 604/187; 604/227

(57) ABSTRACT

A disposable safety syringe has a barrel, a connector assembled at a front end of the barrel, a needle assembly assembled onto the connector, and a plunger extendable into the barrel. The plunger has a connecting head such that the connecting head is able to be securely connected with the connector after an injection via the syringe. Therefore, the needle assembly is able to be pulled by the plunger to fully retract into the barrel and the syringe is then safe to discard without the risk of scratching someone. Moreover, the syringe is provided with a guiding piece to guide the plunger to connect with the connector such that the operation of the syringe is convenient.

### BACKGROUND OF THE INVENTION

1. Field of the Invention

The present invention relates to a syringe and more particularly to a disposable safety syringe. The syringe has a retractable needle to be pulled inside a barrel by a plunger after use, whereby accidental pricking is prevented.

2. Description of Related Art

Healthcare workers routinely have the risk of contracting communicable diseases through accidental exposure to contaminated medical waste products. One of the largest exposure risks to healthcare workers and handlers of the medical waste products is from accidental pricking from used syringe needles. It is reported that odds of a healthcare worker contracting human immunodeficiency virus (HIV) alone through accidental pricking are one in 300. The odds of healthcare workers contracting other serious diseases are even greater.

Therefore a current disposable syringe having a retractable needle is designed to solve the accidental pricking problem. The disposable syringe typically has a retractable needle which is able to be captured by a plunger. By pulling the plunger, the needle is fully retracted inside a barrel, and the contaminated syringe is able to be discarded safely without the risk of pricking someone.

However, the disposable syringe having the retractable needle is able to solve the accidental pricking problem only under an ideal situation. Further problems may still arise in practical use, for example, when retracting the needle, the connection between the needle and the plunger is not secure enough, and may undesirably detach before the needle is fully retracted inside the barrel. The undesirable detachment may cause a health risk to the user due to the chance of accidental pricking.

Accordingly, the present invention tends to provide a disposable safety syringe to mitigate or obviate the aforementioned problems.

### SUMMARY OF THE INVENTION

An objective of the present invention is to provide a disposable safety syringe to prevent accidental pricking, scratching or other harmful exposure to healthcare workers. The syringe has a barrel, a connector assembled at a front end of the barrel, a needle assembly fitted onto the connector, and a plunger extendable into the barrel. The plunger has a connecting head such that the connecting head is able to be received and locked inside the connector after an injection of the syringe so as to connect the plunger and the connector together. Therefore, the needle assembly is able to be pulled by the plunger to fully retract into the barrel and the syringe is then safe to discard without the risk of accidentally scratching someone.

Another objective of the present invention is to provide a disposable safety syringe having the structure as mentioned in the previous objective, wherein the connection between the connector and the connecting head of the plunger is secure and undesirable detachment of the connector and the connecting head during retraction of the needle assembly is prevented.

A further objective of the present invention is to provide a disposable safety syringe having a guiding piece mounted at a rear end of the syringe to guide the plunger so that the connecting head is able to conveniently slide into a chamber defined in the connector.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a disposable safety syringe in accordance with the present invention;

FIG. 2 is an exploded, perspective view of the syringe in FIG. 1;

FIG. 3 is an enlarged, perspective view showing a piston and a connector of the syringe;

FIG. 4 is a cross-sectional view of the syringe, wherein the piston and the connector are not yet connected;

FIG. 5 is a cross-sectional view of the syringe, wherein the piston and the connector are connected together;

FIG. 6 is a cross-sectional view of the syringe after use, wherein a needle assembly is retracted inside a barrel and a plunger is broken off for convenient discarding;

FIG. 7 is a partially enlarged, cross-sectional view showing a state of the syringe such that a connecting head enters a chamber defined in the connector and not yet turned to be locked inside the chamber;

FIG. 8 is a partially enlarged, cross-sectional view showing a further state of the syringe such that the connecting head is turned inside the chamber so as to be locked with the connector;

FIG. 9 is a partially enlarged, cross-sectional view showing a rear end of the syringe, wherein a stem is slidingly guided by a guided piece;

FIG. 10 is a partially enlarged, cross-sectional view showing the rear end of the syringe, wherein the stem is separated from the guided piece;

FIG. 11 is a top view of the syringe under the state shown in FIG. 7; and

FIG. 12 is a top view of the syringe under the state shown in FIG. 8.

### DETAILED DESCRIPTION OF THE PREFERRED EMBODIMENT

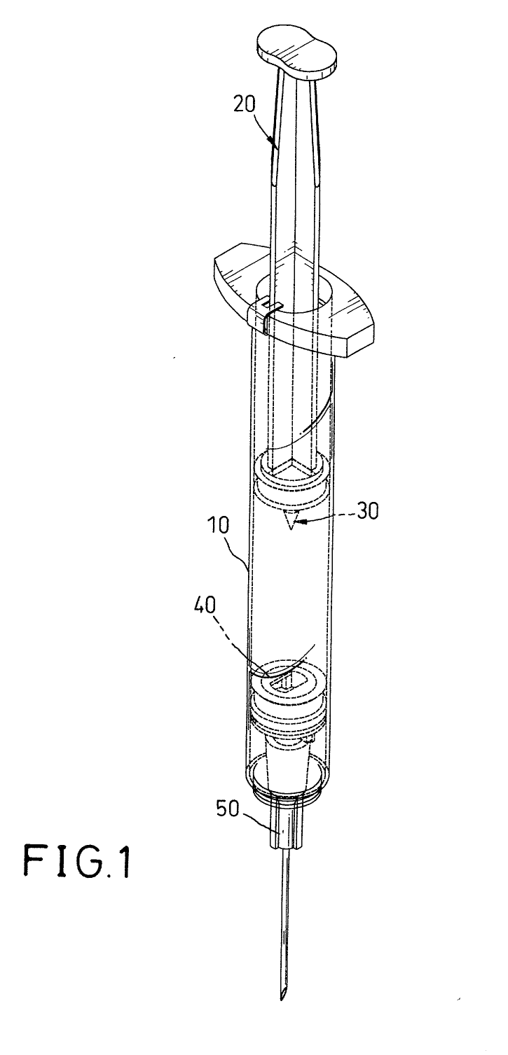

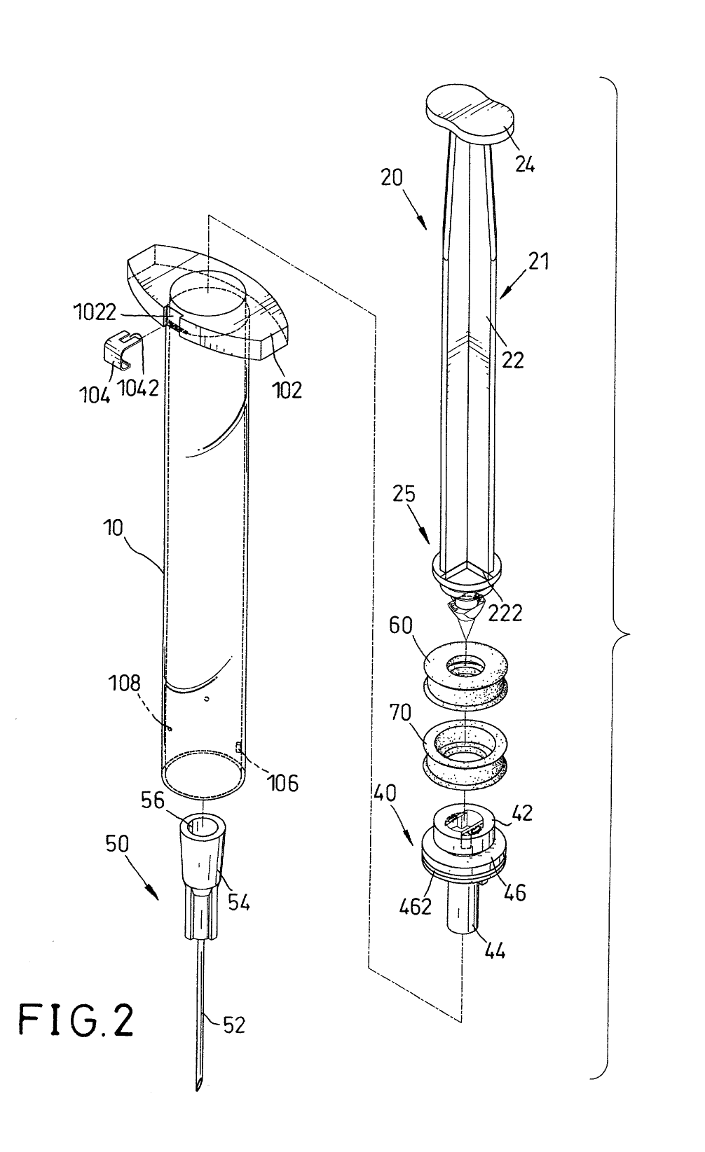

With reference to FIGS. 1 and 2, a disposable safety syringe in accordance with the present invention includes a barrel (10), a plunger (20), a piston (25), a connector (40), a needle assembly (50), an upper sealing ring (60), and a lower sealing ring (70).

The barrel (10) is a hollow cylinder having a front open end and a rear open end. A finger flange (102) is integrally formed at and extending outwardly from the rear open end. A cutout (1022) is defined in a side of a middle section of the finger flange (102). A positioning piece (106) is formed on an inner surface of the barrel (10) and close to the front open end. Multiple retaining protuberances (108) are formed on the inner surface of the barrel (10) and less close to the front open end with respect to the positioning piece (106).

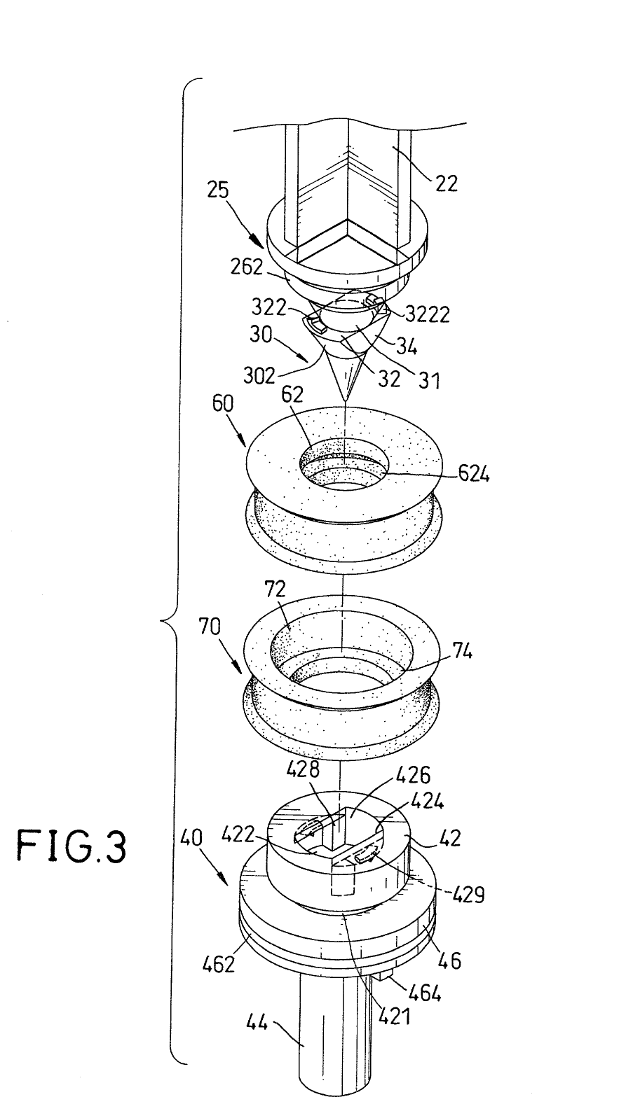

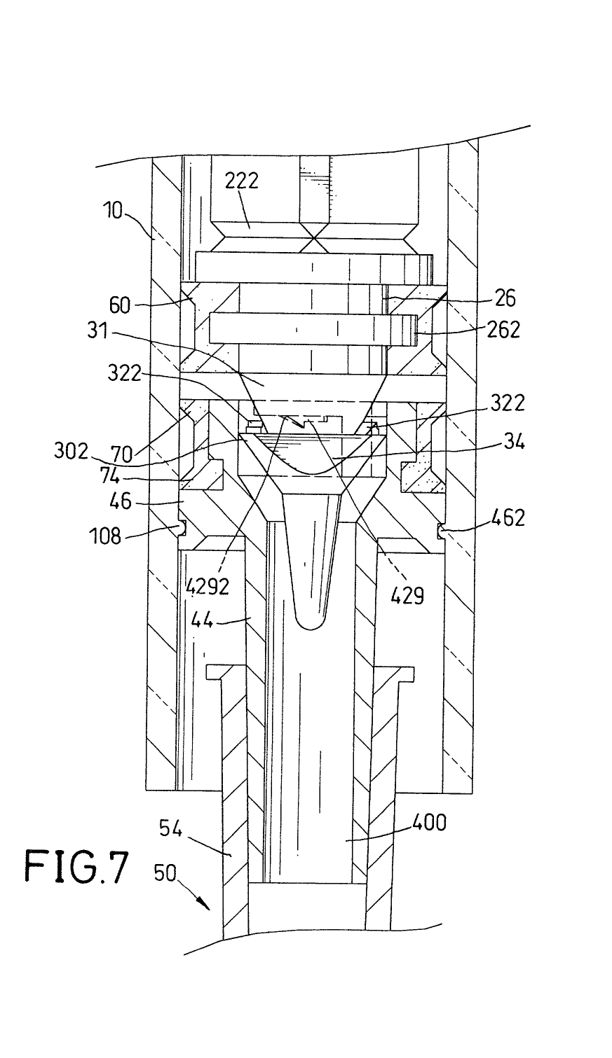

With reference to FIGS. 2 and 3, the plunger (20) includes a stem (21), a thumb pad (24) formed at a rear end of the plunger (20), and the piston (25) formed at a front end of the plunger (20). The stem (21) is made of multiple elongated body pieces (22) which are tapered inward near the rear end of the plunger (20). Each one of the body pieces (22) is provided with a fracture line (222) defined near a front end of the plunger (20) so as to allow the body pieces (22) to be easily broken off and thus separated from the piston (25) after completion of the injection process. Referring to FIGS. 3 and 7, the piston (25) includes a retaining portion (26), a connecting head (30), and a neck (31) formed between the retaining portion (26) and the connecting head (30). An annular flange (262) is formed around the retaining portion (26). The connecting head (30) is configured as a cone and has two shoulders (302) oppositely formed. Each one of the shoulders (302) has a top surface (32). Two first stops (322) are respectively formed on the top surfaces (32) and pointing in the opposite directions. Each one of the first stops (322) has a first slope (3222). The connecting head (30) has opposite sides shaped as two flat walls (34).

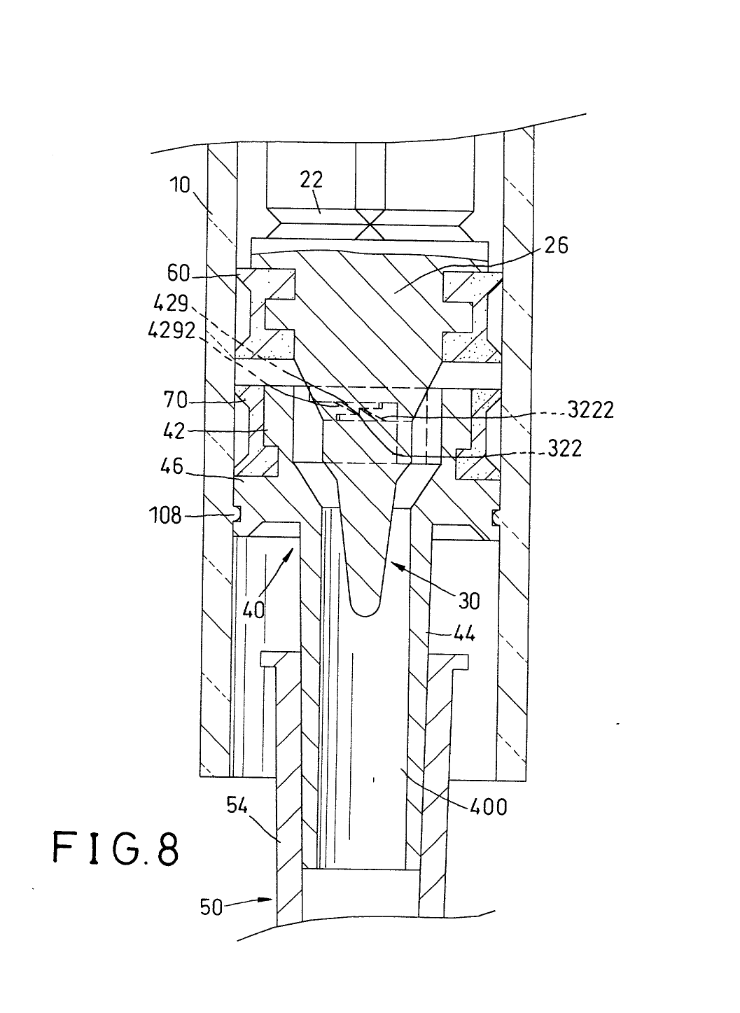

The connector (40) is hollow, and includes a cylinder portion (42), a tube portion (44) and a disk portion (46) formed between the cylinder portion (42) and the tube portion (44). A chamber (422) is defined inside the cylinder portion (42) to receive the connecting head (30) and to allow the connecting head (30) to rotate inside the chamber (422). Two columns (428) are oppositely formed in the chamber (422) so as to restrict the range and the direction that the connecting head (30) is able to rotate. Two ceilings (424) are formed on a top surface of the cylinder portion (42) and extend toward each other. An opening (426) is defined between the ceilings (424) and communicates with the chamber (422) such that the connecting head (30) is able to enter the chamber (422) via the opening (426) while the flat walls (34) slide over edges of the ceilings (424). Two second stops (429) complementary with respect to the first stops (322) are formed on inner surfaces of the ceiling (424), and point in opposite directions. Two second slopes (4292) (shown in FIG. 8) are respectively formed on the second stops (429). A receiving groove (421) is defined around the cylinder portion (42) and a retaining groove (462) is defined around the disk portion (46). An abutting piece (464) is formed on a bottom surface of the disk portion (46) to engage with the positioning piece (106) of the barrel (10).

Referring to FIG. 2, the needle assembly (50) to be mounted onto the connector (40) has a hollow needle (52) extending out from a needle hub (54), wherein the hollow needle (52) and the needle hub (54) are securely connected together. A receiving hole (56) configured for receiving the tube portion (44) of the connector (40) is longitudinally defined in the needle hub (54).

The upper sealing ring (60) has a through bore (62) to be mounted around the retaining portion (26) of the piston (25). An annular groove (624) is defined in an inner surface defining the through bore (62) to securely receive the annular flange (262) of the retaining portion (26).

The lower sealing ring (70) has a through hole (72) to be mounted around the cylinder portion (42) of the connector (40). An inward flange (74) is formed on an inner surface defining the through hole (72) to be securely received inside the receiving groove (421) defined in the cylinder portion (42).

With reference to FIGS. 1 and 7, when assembling the syringe, the lower sealing ring (70) is securely mounted onto the connector (40) by extending the cylinder portion (42) into the through hole (72) and securely receiving the inward flange (74) in the receiving groove (421). The connector (40) is then assembled into the front open end of the barrel (10) by fitting the protuberances (108) in the retaining groove (462), while the positioning piece (106) of the barrel (10) engages with the abutting piece (464) of the connector (40). The upper sealing ring (60) is mounted onto the piston (25) by extending the retaining portion (26) of the piston (25) into through bore (62) defined in the upper sealing ring (60) and receiving the annular flange (262) into the annular groove (624). The plunger (20) extends the piston (25) into the barrel (10) from the rear open end and a guiding piece (104) having a notch (1042) (shown in FIG. 2) is mounted into the cutout (1022) defined in the finger flange (102), while the appropriate one of the body pieces (22) extends through the notch (1042). Finally the needle assembly (50) is mounted onto the connector (40) by force fitting the tube portion (44) into the receiving hole (56) defined in the needle hub (54).

The procedure of performing an injection using the syringe is same as those of the prior art syringes, and owing to that syringes are widely known medical instruments and have been used for years, therefore excessive description on the injection procedure of the syringe is omitted.

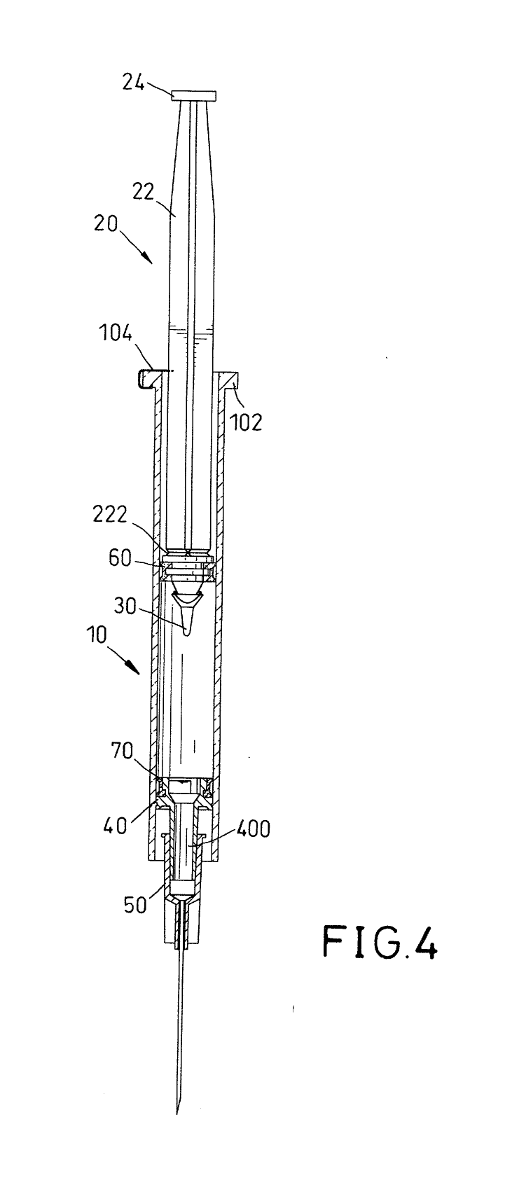

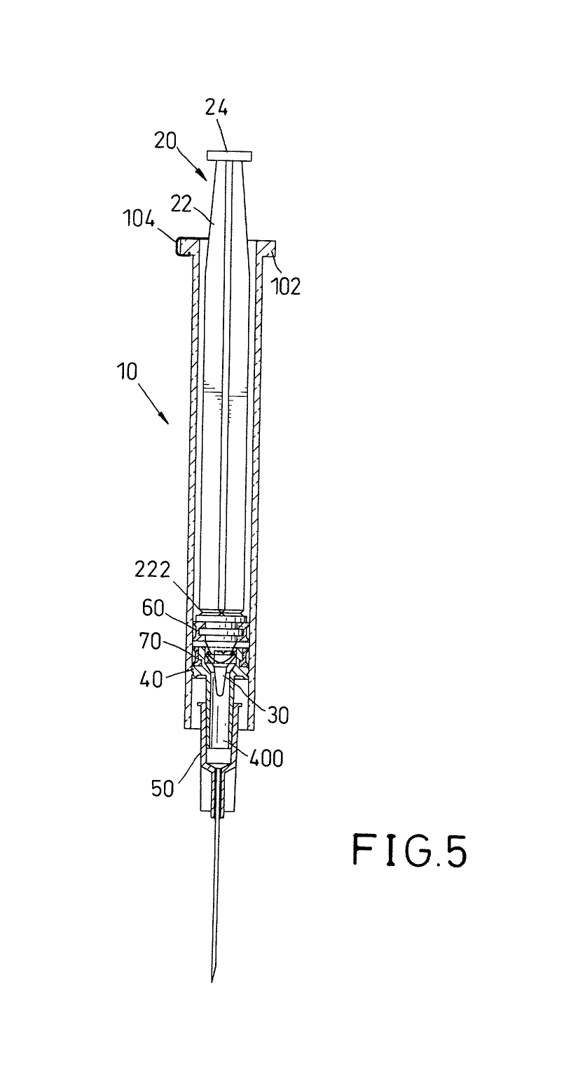

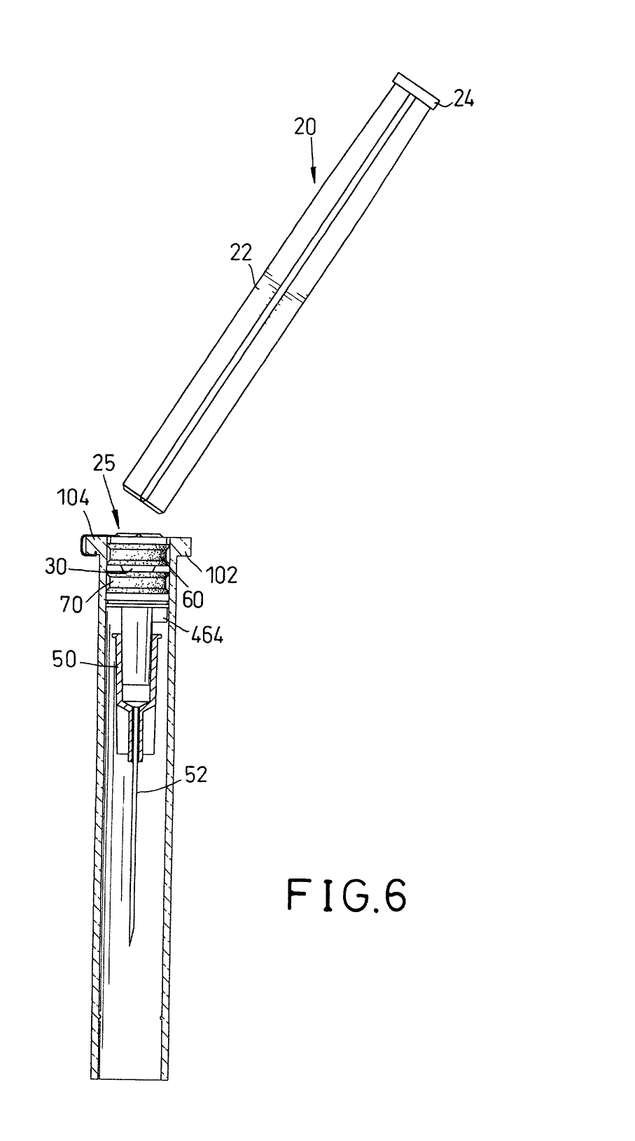

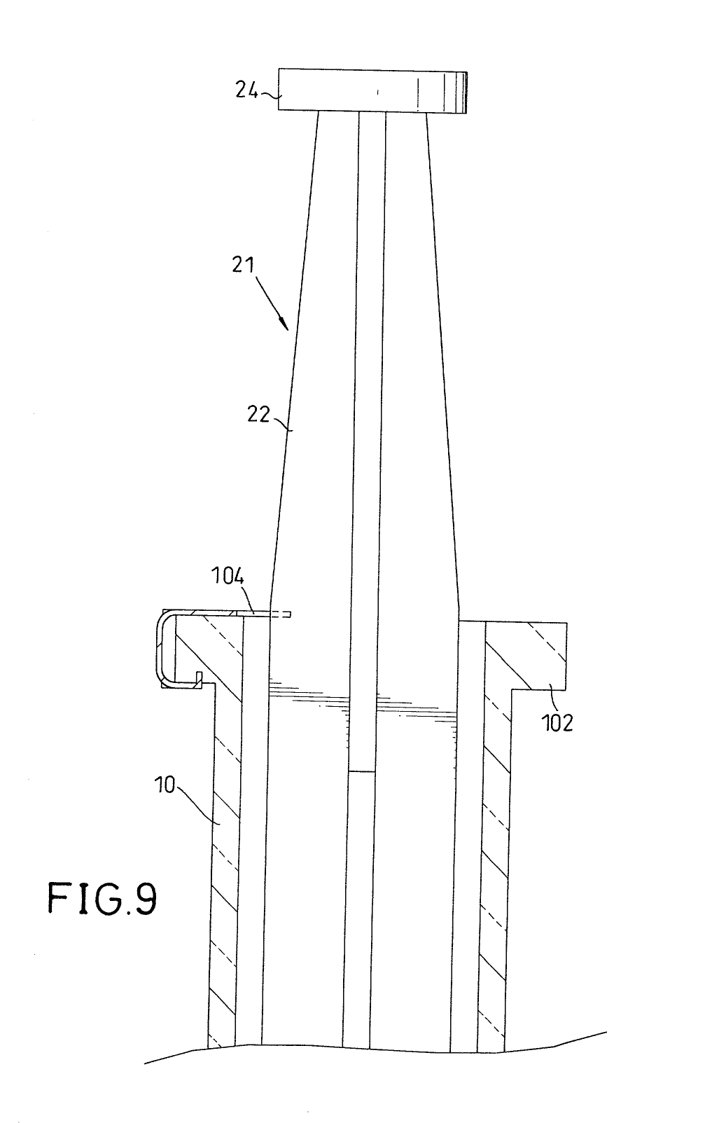

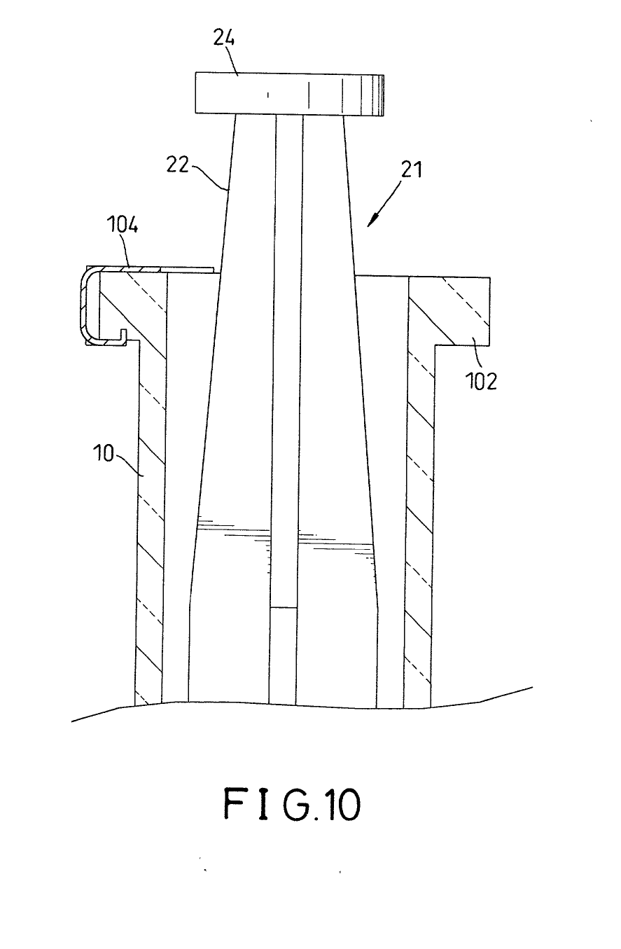

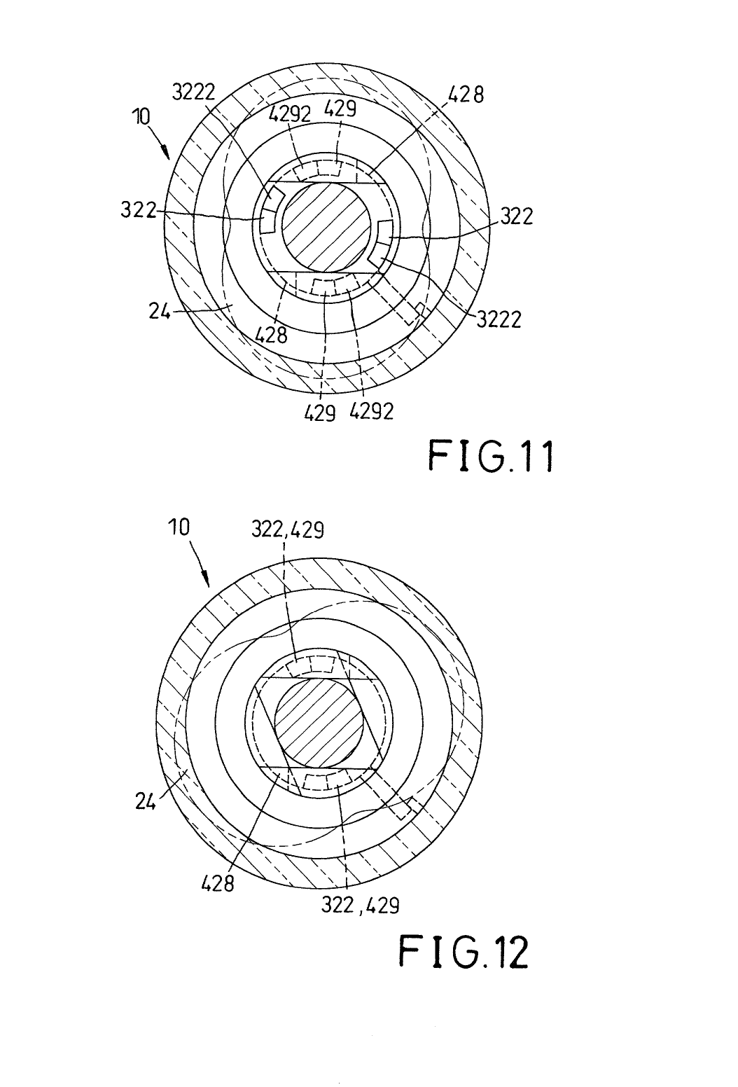

With reference to FIGS. 3, 4 and 5, after use of the syringe, the plunger (20) is pushed further into the barrel (10) so as to connect the connecting head (30) with the connector (40). During the movement of the plunger (20), the body piece (22) is slid through and restricted in the notch (1042) (shown in FIG. 2) defined in the guiding piece (104) and therefore guides the plunger (20) so that the connecting head (30) aligns with the opening (426) defined in the connector (40), wherein the position of the connector (40) is determined by the engagement between the positioning piece (106) and the abutting piece (464). The plunger (20) is further pushed and enters the chamber (422) via the opening (426) while the flat walls (34) slide over the edges of the ceiling (424). At this moment, the connector (40) and the connecting head (30) are at a state as shown in FIG. 7. With reference to FIGS. 9 and 10, it is noted that the guiding piece (104) guides the stem (21) until the tapered rear end of the body piece (22) separates from the guiding piece (104). With reference to FIGS. 11 and 12, when the stem (21) is not restricted, the plunger (20) is able to be turned in a clockwise direction from a first position shown in FIG. 11 to a second position shown in FIG. 12, and the connector (40) remains stationary due to the friction provided between the lower sealing ring (70) and the inner surface of the barrel (10). When turning from the first position to the second position, the first slopes (3222) respectively slide over the second slopes (4292) and therefore the first tops (322) engage with the corresponding second stops (429) to securely connect the connecting head (30) with the connector (40). With reference to FIG. 6, finally by pulling the plunger (20), the connector (30) together with the needle assembly (50) is fully retracted inside the barrel (10), and the body piece (22) is broken off to separate from the piston (25). Accordingly, the contaminated needle (52) is completely received inside the barrel (10) and the used syringe is able to be discarded safely without accidentally pricking someone.

From the above description, it is noted that the invention has the following advantages:

1. secure connection. The connection between the connecting head (30) and the connector (40) is secure and undesirable detachment during the retraction of the needle assembly (50) is prevented.

2. convenient operation. The syringe is equipped with the guiding piece (104), whereby the plunger (20) is able to be automatically aligned with the opening (426) of the connector (40) to enter the chamber (422) in order to provide a convenient operation of the syringe.

While this invention has been particularly shown and described with references to the preferred embodiment thereof, it will be understood by those skilled in the art that various changes in form and details may be made therein without departing from the scope of the invention encompassed by the appended claims.

What is claimed is:

1. A disposable safety syringe comprising:
a hollow barrel having a front open end and a rear open end;
a plunger slidably extendable into the hollow barrel from the rear open end and having a front end to extend into the hollow barrel, an upper sealing ring securely mounted around the front end so as to engage with an inner surface of the hollow barrel in a fluid-tight manner and a connecting head formed at the front end of the plunger and provided with at least two shoulders oppositely extending outward and each of the at least two shoulders having a first stop formed on a top face of the at least two shoulders;
a connector being hollow and mounted inside the front open end of the barrel, the connector having a cylinder portion and a tube portion, the cylinder portion defining a chamber to correspond to the connecting head and an opening communicating with the chamber so that the connecting head is able to enter the chamber via the opening, at least two second stops oppositely formed on an inner periphery defining the chamber to complementarily correspond to the at least two first stops of the connecting head so that after the connecting head is inserted into the chamber and rotated, the engagement between the at least two first stops and the complementary at least two second stops allows the connecting head to be securely connected with the connector; and
a lower sealing ring securely mounted around the cylinder portion of the connector so as to engage with the inner surface of the hollow barrel in the fluid-tight manner,
whereby after an injection process using the disposable safety syringe is completed, the connecting head is able to be securely connected with the connector so that the needle assembly is able to be fully retracted inside the barrel by pulling the plunger with out the risk of accidentally pricking someone.

2. The disposable safety syringe as claimed in claim 1 further comprising a finger flange outwardly formed at the rear open end of the barrel and a guiding piece securely mounted onto the finger flange, wherein a notch is defined in the guiding piece and the plunger has multiple elongated body pieces extending through the notch so as to be slidingly guided in the notch.

3. The disposable safety syringe as claimed in claim 2 further comprising a disk portion formed between the cylinder portion and the tube portion, wherein the disk portion is provide with an abutting piece engaging a positioning piece formed in the inner surface of the barrel close to the front open end to provide positioning of the connector.

4. The disposable safety syringe as claimed in claim 3 further comprising two columns oppositely formed in the chamber to restrict the range and the direction which the connecting head is able to rotate.

5. The disposable safety syringe as claimed in claim 3, wherein an annular groove is defined around the disk portion and multiple protuberances are formed in the inner surface of the barrel close to the front open end such that by securely receiving the multiple protuberances in the annular groove, the connector is mounted onto the barrel.
